(19) United States
(12) Patent Application Publication
(10) Pub. No.: US 2009/0312593 A1
(43) Pub. Date: Dec. 17, 2009

(54) Radioactive Device for treatment of a Tumor

(76) Inventors: Christopher D. Drobnik, Wauconda, IL (US); Michael W. Drobnik, Downers Grove, IL (US)

Correspondence Address: Carmen Patti Law Group , LLC, ONE N. LASALLE STREET, 44TH FLOOR, CHICAGO, IL, 60602, US

(21) Appl. No.: 12/224,640

(22) PCT Filed: Mar. 14, 2007
(86) PCT No.: PCT/US2007/006421

§ 371 Date: Sep. 2, 2008

Related U.S. Application Data
(60) Provisional application No. 60/781,728, filed on Mar. 14, 2006.

(51) Int. Cl. A61M 36/04

(52) U.S. Cl. 600/3

(57) ABSTRACT

An apparatus in one example has a treatment balloon or similar device that may be implanted into a tumor bed or cavity, and filled with or carrying a radioactive material.

### TECHNICAL FIELD

The invention relates generally to treatment of tumors, and more particularly to a device for applying radiation to a tumor bed.

### BACKGROUND

Radiation therapy is an accepted method, either independently or in combination with another therapy, to treat breast cancer, for example. Traditional means involve the use of external beam radiation therapy or brachytherapy, whereby catheters are temporarily placed into the breast to receive a radiation source during the course of a short (1-6 week) treatment period.

There has been a move to partial breast irradiation therapy following breast conservation surgery in recent years, primarily led by Cytyc Corporation and their balloon-based MammoSite applicator. Xoft Microtube has a similar product that uses an electronic X-ray source in place of the traditional radioactive source employed by Proxima.

Even with the current market acceptance of such novel products introduced for breast brachytherapy, there is still a need for further product improvements to minimize cost, improve patient convenience and comfort, and minimize potential side effects from the delivery of therapeutic amounts of radiation. One need in the field is for a radiation delivery vehicle that could be introduced following tumor removal and that requires minimal physician or patient intervention after implantation. The Proxima and Xoft techniques use traditional high dose rate brachytherapy. It is a drawback that the implanted catheters must be left in place for about one week and a radioactive source is placed in them twice daily for about 10 minutes for 7 straight days. The catheters are then removed.

Thus, there is a need in the prior art to overcome the present disadvantages of existing apparatus and methods for brachytherapy and similar procedures.

### SUMMARY

The invention in one implementation encompasses an apparatus. The apparatus may comprise: a treatment balloon or similar device that may be implanted into a tumor bed (also referred to as a cavity) and filled with or carrying a radioactive material.

The invention in another implementation encompasses a method. This embodiment of the method may comprise: providing a treatment balloon, gel, foam, or similar device with a radioactive material; and implanting the device in a tumor bed (also referred to as a cavity).

### DESCRIPTION OF THE DRAWINGS

Features of exemplary implementations of the invention will become apparent from the description, the claims, and the accompanying drawings in which:

FIG. 1 depicts one embodiment of the present method and apparatus that may be referred to as a gel-filled balloon.

FIG. 2 depicts one embodiment of the present method and apparatus that may be referred to as a modified double balloon.

FIG. 3 depicts one embodiment of the present method and apparatus that may be referred to as a balloon applicator.

FIG. 4 depicts another view of the FIG. 3 balloon applicator.

FIG. 5 depicts one embodiment of the present method and apparatus that may be referred to as a seed balloon.

FIG. 6 depicts one embodiment of the present method and apparatus that may be referred to as a solid carrier.

FIG. 7 depicts one embodiment of the present method and apparatus that may be referred to as an amorphous radiation carrier delivery.

FIG. 8 depicts a further embodiment of the FIG. 7 embodiment.

FIG. 9 depicts a further embodiment of the FIG. 7 embodiment.

FIG. 10 depicts one embodiment of the present method and apparatus that may be referred to as a dual isotope treatment.

FIG. 11 depicts one embodiment of the present method and apparatus that may be referred to as a cavity scaffold.

FIG. 12 depicts one embodiment of the present method and apparatus that may be referred to as a cavity filament.

FIG. 13 depicts one embodiment of the present method and apparatus that may be referred to as an autologous fat treatment.

### DETAILED DESCRIPTION

Malignant tumors, when surgically resected, leave excavated cavities of various dimensions and configurations in the operated area, such as a breast. Because of the present day inability to definitively surgically eradicate all cancer-bearing tissue at the site of operation, radiation treatment is delivered to the site of surgical excision to incorporate, for example, a 2-3 cm margin of normal appearing tissue on all sides of the surgical cavity. However, the geometry of the cavity is predicated on the particular geometry of the resected breast tumor, for example, and, because of this, is usually irregular in geometric shape. Because of this, the delivery of radiation treatment to the unmodified surgical margin cannot be uniformly delivered utilizing present day brachytherapy delivery systems. The following embodiments overcome this deficiency in the prior art and may be applicable for use in cavities that surgically occur due to a variety of reasons.

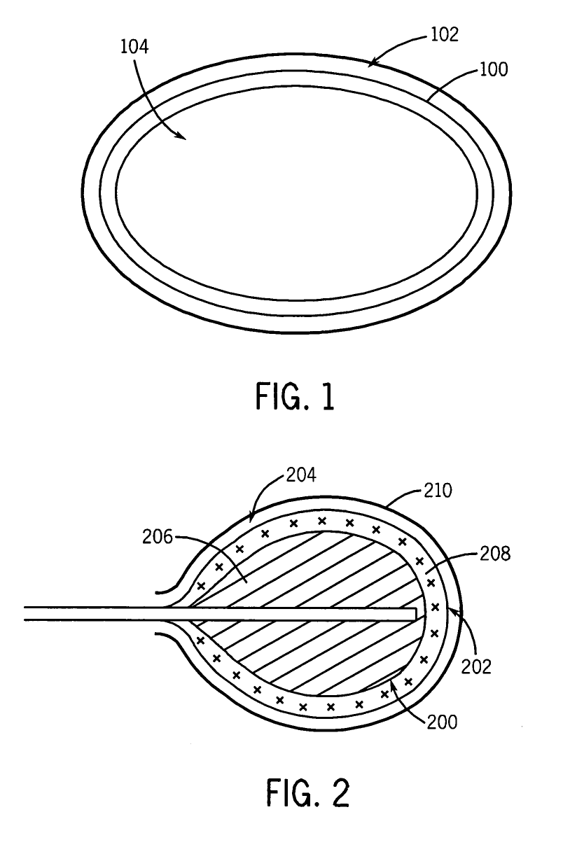

As depicted in FIG. 1, one embodiment of the present method and apparatus may be referred to as a gel-filled balloon. In this embodiment a treatment balloon 100 may be implanted into a tumor bed 102 (also referred to as a cavity) and filled with a radioactive material 104 that may be a gel or a liquid. The radioactive material 104 may act as a radiation carrier, as well as provide support for the tumor bed 102 and act as a replacement for removed tissue. The balloon 100 may remain in the cavity 102 permanently, or resorb over time. The balloon 100 may be rigid and define the shape of the cavity 102, or be flexible to conform to the shape of the cavity 102. If the balloon is designed to be resorbable, the contained radioactive material 104 may also resorb over time and be replaced by natural tissue, or the radioactive material 104 may remain as a permanent structure. Thus, the balloon 100 may or may not be bio-absorbable, and the radioactive material 104 may or may not be bio-absorbable. All of these combinations are within the scope of the embodiments of the present method and apparatus. The “balloon” may also comprise a material that is expanded within the cavity to conform to or modify the shape of the cavity. Suitable non-limiting examples of such materials include screens and meshes.

In general, embodiments of the present method and apparatus have as a feature that the radioactive material may flow into the balloon, for example, in the cavity and take the natural shape of the cavity, thus substantially mimicking what was removed so that the tissue may be built back up to what was normal. After the balloon is inserted into the cavity, the balloon may be formed to the cavity via any appropriate means, such as suction or pressure.

As depicted in FIG. 2, one embodiment of the present method and apparatus may be referred to as a modified double balloon. In this embodiment, an inner balloon 200 and an outer balloon 202 may be implanted into a tumor bed 204. The inner balloon 200 may contain an inert material 206, such as saline, and act as a tissue support. The radioactive material 208 may be disposed within a skin of the inner balloon 200, in space created between the inner and outer balloons 200, 202, or in a skin of the outer balloon 202. In this way, the therapeutic isotope 208 may be localized only near the tumor bed walls 210, and may limit the amount of isotope 208 needed to treat the tumor bed 204. As opposed to the gel-filled balloon depicted in FIG. 1, this embodiment may not have any radiation emanating from the center of the implant, which is comprised of the inert material 206. This minimizes the use of radioactive material, which would be greatly shielded by the bulk of the implant before it reaches the tumor bed walls.

As one example, the radioactive gel implant may have a diameter of about 4 cm. It can be advantageous for the radioactive material to be at or in contact with the tumor wall. Thus, to accomplish this only the outer area between the two balloons has the radioactive material, the inside of the inner balloon having only a filler material. This inner balloon may then be filled with saline or silicone. The gap between the inner and outer balloon may be small, for example about 5 mm or less. Thus a minimum amount of radioisotope is used in this embodiment.

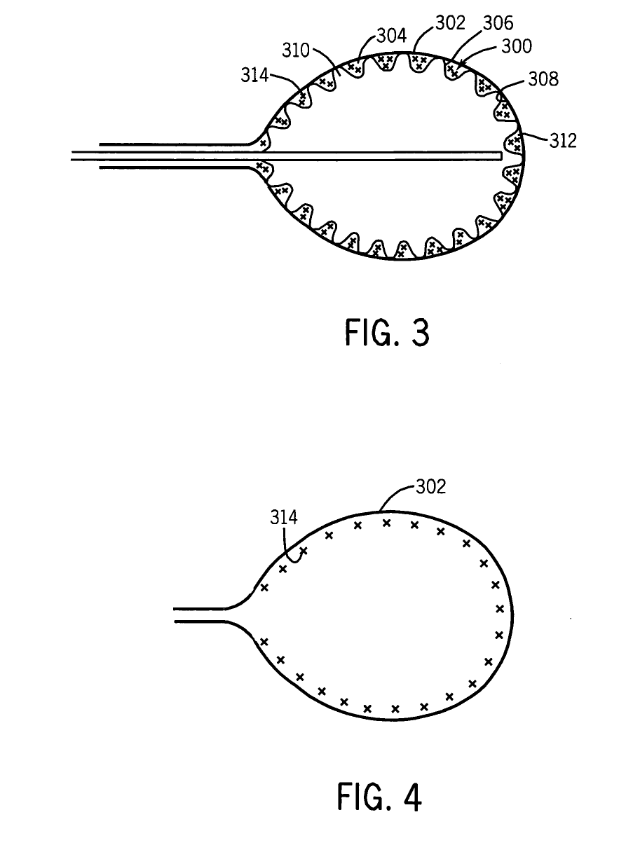

As depicted in FIGS. 3 and 4, one embodiment of the present method and apparatus may be referred to as a balloon applicator. In this embodiment a modified balloon 300 is placed in a tumor bed 302 to allow for the dispensing of a radioactive material 304 between an outer surface 306 of the balloon 300 and the wall 308 of the tumor bed 302. The balloon 300 may have protrusions 310 on its surface 306 which act to create a space 312 between the balloon 300 and the wall 308 of the tumor bed 302. The resulting space 312 may then be filled with a radioactive material 314. The balloon 300 may have various configurations such as dimpled configuration (like a golf ball), or a channeled configuration whereby channels are disposed over the balloon surface to create the described space 312. The radioactive material 314 may be dispensed with a tissue glue, adhesive, or solidifying agent for example, so that it adheres to the tissue surface of the wall 308 of the tumor bed 302. The balloon 300 may then be removed or be absorbed by the body, yielding a “skin” of radioactivity behind in a predetermined pattern as depicted in FIG. 4. Alternatively, the balloon could remain in place permanently. The balloon in this embodiment does not have to have an attached catheter.

The radioactive material 304 may take the form of a seed that may be formed of titanium and that may have the size of a grain of rice, for example. More specifically, the radioactive material, commonly an isotope of iodine or palladium, may be enclosed in small stainless steel, titanium, or plastic shells which may have a diameter of about 0.8 mm and a length of about 4.5 mm. Alternatively, the radioactivity could be in the form of a particle, gel, foam, or solidified liquid.

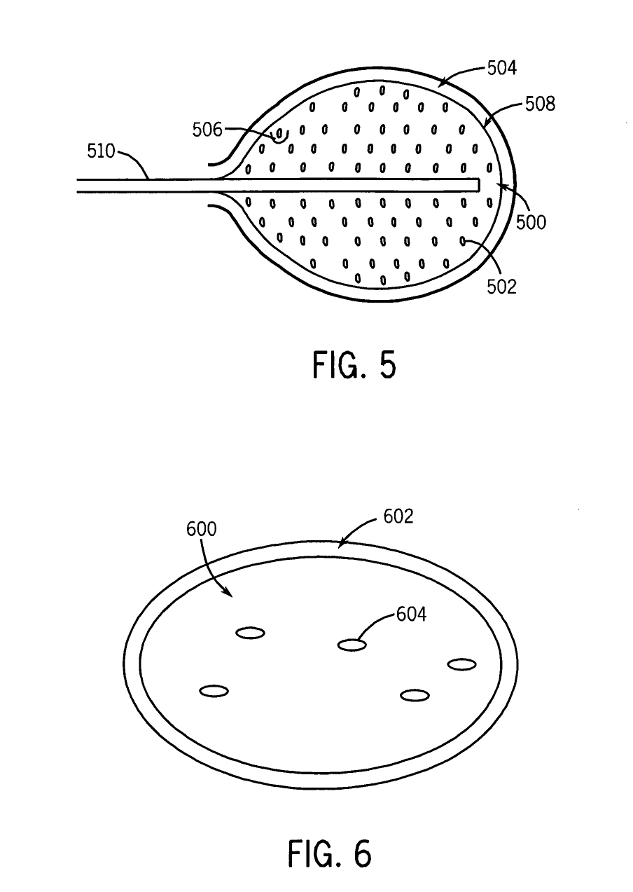

As depicted in FIG. 5, one embodiment of the present method and apparatus may be referred to as a seed balloon. In this embodiment, a modified balloon 500 is used to hold radioactive seeds 502 in place within the cavity 504. The balloon 500 may have pockets 506 in which the radioactive seeds 502 are placed, or the radioactive seeds 502 may be adhered to a wall 508 of the balloon 500. The balloon may be permanent, removable, or resorbable. The balloon may be inserted and inflated via a catheter 510, for example.

As depicted in FIG. 6, one embodiment of the present method and apparatus may be referred to as a solid carrier. In this embodiment, following for example a lumpectomy, a pliable solid 600 may be molded to fit the resulting cavity 602. This solid 600 may then be removed, loaded with a radioactive material 604, then placed back in the tumor bed or cavity 602. The radioactive material 604 may be in the form of common seeds, or as a liquid, solid, gel, filament, pellet or other radiation carrier. The molded solid 600 may be resorbable, or remain in place permanently to provide support for the surrounding tissue.

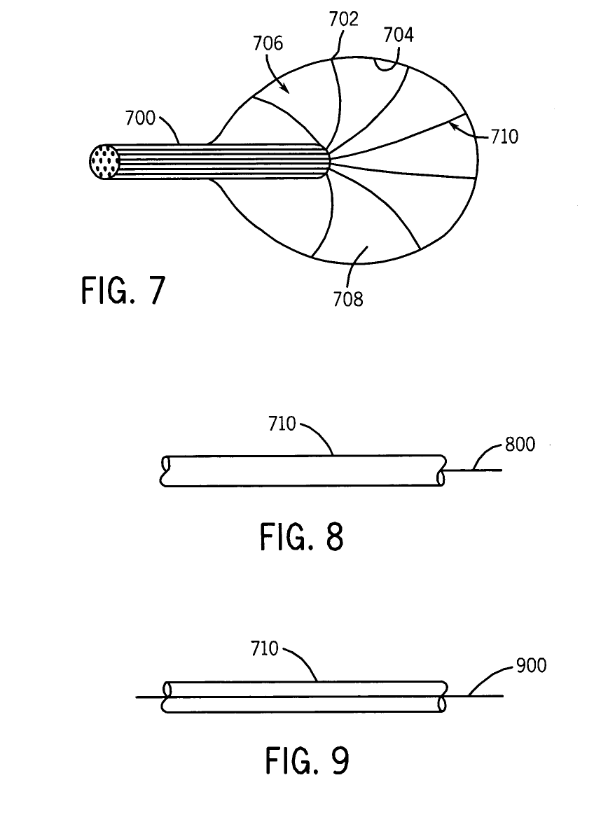

As depicted in FIG. 7, one embodiment of the present method and apparatus may be referred to as an amorphous radiation carrier delivery. In this embodiment, an applicator 700 may allow for injection of a radioactive material 702 into a wall 704 of a tumor bed or cavity 706. A solid structure 708, approximating the size of the tumor bed 706, may be placed within the excised cavity 706. This structure may have numerous channels or ports 710 which may allow access to the exterior of the structure 708, and hence to the wall 704 of the cavity 706. The radioactive material 702 may be introduced through these channels 710 into the tissue surrounding the structure 708. To accomplish the injection of the radioactive material 702, the channels 710 may be fitted with needle-like projections 800 (see FIG. 8), or the channels 710 may be designed to allow the passage of a flexible needle 900 (see FIG. 9) filled with the radioactive material 702. Using such an apparatus the tumor wall 704 may be treated one or more times with radioactive material 702 in a way analogous to piercing the wall of an inflated balloon from the inside out.

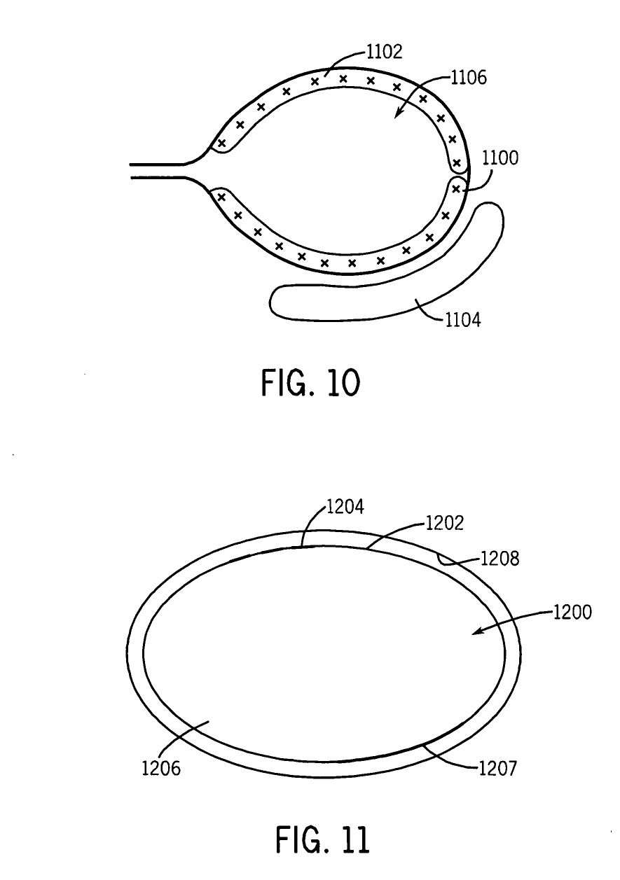

As depicted in FIG. 10, one embodiment of the present method and apparatus may be referred to as a dual isotope treatment. In this embodiment two or more isotopes, such as isotopes 1100 and 1102 may be used simultaneously with any of the above-described embodiments in order to protect critical structures, such as structure 1104 near a tumor bed 1106. For example, a low-energy isotope 1100 may be used in an implant where the implant comes in contact with or is near an organ, such as structure 1104, which is sensitive to radiation. A higher energy isotope 1102 may be used in the rest of the implant where the risk of collateral damage to neighboring organs is not as great.

As depicted in FIG. 11, one embodiment of the present method and apparatus may be referred to as a cavity scaffold. In this embodiment, once a tumor has been removed the remaining cavity 1200 may be supported with a stent-type device 1202. The stent-type device 1202 may be loaded with radioactive material 1204 to provide a therapeutic dose to the tumor margin. Alternatively, the supported cavity 1200 may be filled with a radioactive substance 1206. Alternatively, the stent-type device 1202 may be used to hold a radioactive film or sheet 1207 in place against a wall 1208 of the cavity 1200.

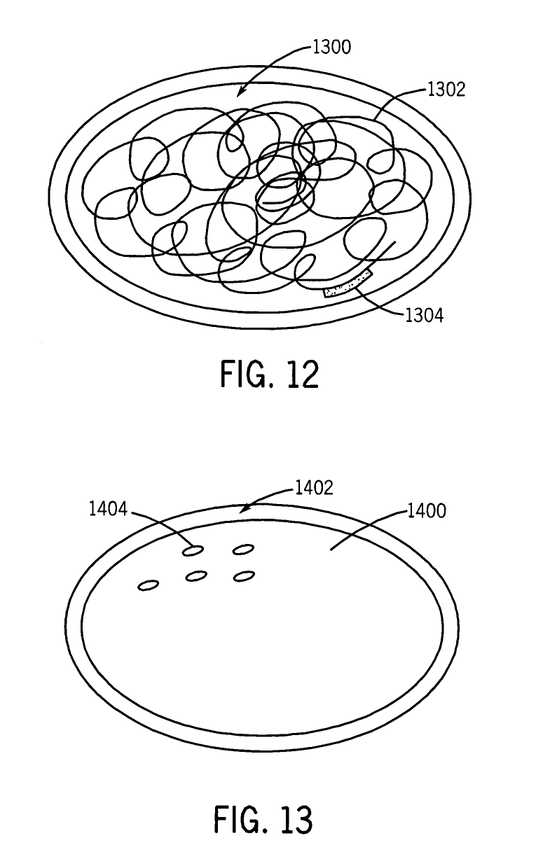

As depicted in FIG. 12, one embodiment of the present method and apparatus may be referred to as a cavity filament. In this embodiment once a tumor has been removed the remaining cavity 1300 may be filled with a flexible radioactive filament 1302 (the resulting structure may look like a ball of yarn). The filament 1302 itself may be radioactive, or be co-dispensed with a radioactive substance 1304 and serve only to fill the cavity 1300.

As depicted in FIG. 13, one embodiment of the present method and apparatus may be referred to as an autologous fat treatment. In this embodiment fat 1400 may be harvested from the body for implantation into a tumor cavity 1402. The fat 1400 may be radiolabeled, or be doped with radioactive particles or radioactive compounds 1404.

The above-described embodiments may use various biological adhesives, including tissue glue. A tissue glue is a natural biological material that takes advantage of natural components of the human clotting system. The clotting proteins and cofactors are either extracted from donor blood, or extracted from the patient's own blood prior to surgery, or, in many cases, extracted from the patient's own blood during the surgery itself. Typically, the glue is maintained in two components, one with fibrin protein solution and the other with, e.g., the calcium solution which helps activate the clotting cascade. The two components are loaded into two separate syringes and the needle tips from the two syringes are bent to run parallel to each other so that the two tips are closely apposed. The surgeon uses a double syringe apparatus to apply the two fluids to the surface of interest simultaneously. As the two fluids emerge from the needle tips onto the tissue surface they mix and congeal.

The above-described embodiments may also incorporate radiation-shielding materials into their construction to directionally modify the emitted radiation. For example, the region of a device which approaches an anatomical structure sensitive to radiation could be fitted with shielding material to lessen the radiation exposure to the sensitive structure. The shielding material could take the form of sheets or particles comprised of metals, barium, bismuth, or polymers impregnated with said shielding materials.

Although exemplary implementations of the invention have been depicted and described in detail herein, it will be apparent to those skilled in the relevant art that various modifications, additions, substitutions, and the like can be made without departing from the spirit of the invention and these are therefore considered to be within the scope of the invention as defined in the following claims.

We claim:

1. An apparatus, comprising:
a treatment structure associated with a cavity corresponding to a tumor bed; and
a radioactive material associated with the treatment structure.

2. The apparatus according to claim 1, wherein the treatment structure is a treatment balloon implantable in the cavity, and wherein the radioactive material fills the treatment balloon.

3. The apparatus according to claim 2, wherein radioactive material is one of a gel and a liquid.

4. The apparatus according to claim 2, wherein The radioactive material functions as a radiation carrier, a support for the tumor bed and as a replacement for tissue that is removed to form the tumor bed.

5. The apparatus according to claim 2, wherein the balloon is one of bio-absorbable and non-bio-absorbable, and wherein the radioactive material is one of bio-absorbable and non-bio-absorbable.

6. The apparatus according to claim 2, wherein the balloon is one of rigid and defines a shape of the tumor bed, and flexible to conform to a shape of the tumor bed.

7. The apparatus according to claim 2, wherein the balloon is an inflated mesh.

8. The apparatus according to claim 2, wherein the treatment balloon is a modified double balloon having an inner balloon and an outer balloon.

9. The apparatus according to claim 8, wherein the inner balloon contains an inert material and acts as a tissue support.

10. The apparatus according to claim 8, wherein the radioactive material is placed in one of a skin of the inner balloon, in a space created between the inner and outer balloons, and a skin of the outer balloon.

11. The apparatus according to claim 2, wherein the treatment balloon is a modified balloon placeable in a tumor bed to allow for dispensing a radioactive material between an outer surface of the balloon and the wall of the tumor bed, and wherein the balloon has protrusions on a surface thereof which creates a space between surface of the balloon and a wall of the tumor bed, and wherein the space is filled with the radioactive material.

12. The apparatus according to claim 11, wherein the radioactive material is dispensed with a tissue glue so that the radioactive material adheres to a tissue surface of the wall of the tumor bed.

13. The apparatus according to claim 11, wherein when the balloon is removed, a skin of radioactive material is left behind in a predetermined pattern on the tissue surface of the wall of the tumor bed.

14. The apparatus according to claim 2, wherein the treatment balloon is a modified balloon that holds radioactive seeds in place within the tumor bed, the balloon having one of pockets in which the radioactive seeds are placed, and the radioactive seeds being adhered to a wall of the balloon.

15. The apparatus according to claim 2, wherein the radioactive material has at least first and second isotopes, and wherein the first isotope is distributed in at least one first area of the tissue surrounding the cavity, and wherein the second isotope is distributed in at least one second area of the tissue surrounding the cavity.

16. The apparatus according to claim 1, wherein the treatment structure is a pliable solid that is molded to fit the cavity, and wherein the radioactive material is contained in the pliable solid.

17. The apparatus according to claim 16, wherein the radioactive material comprises at least one of common seeds, a liquid, a solid, a gel, a filament, and a pellet.

18. The apparatus according to claim 16, wherein the pliable solid is one of bio-absorbable and non-bio-absorbable, and wherein the radioactive material is one of bio-absorbable and non-bio-absorbable.

19. The apparatus according to claim 16, wherein the radioactive material has at least first and second isotopes, and wherein the first isotope is distributed in at least one first area of the tissue surrounding the cavity, and wherein the second isotope is distributed in at least one second area of the tissue surrounding the cavity.

20. The apparatus according to claim 1, wherein the treatment structure is a solid structure approximating a size of the cavity;
wherein a plurality of passages in the solid structure extend from a common port to a respective plurality of ports at an outer surface of the solid structure; and
wherein the radioactive material is injected into tissue that surrounds the cavity, the radioactive material being introducible to the tissue via the plurality of passages and respective ports.

21. The apparatus according to claim 20, wherein the radioactive material has at least first and second isotopes, and wherein the first isotope is distributed in at least one first area of the tissue surrounding the cavity, and wherein the second isotope is distributed in at least one second area of the tissue surrounding the cavity.

22. The apparatus according to claim 1, wherein the treatment structure is a stent-type device, and wherein the radioactive material is at least one of radioactive cavity fill substance, and a radioactive film that is held against the tissue by the stent-type device.

23. The apparatus according to claim 1, wherein the treatment structure is a flexible filament, and wherein the radioactive material is at least one of radioactive filament, and a radioactive substance that adheres to the flexible filament.

24. The apparatus according to claim 1, wherein the treatment structure is fat may be harvested from a corresponding body, and wherein the radioactive material is at least one of radiolabeled fat, radioactive particles, and radioactive compounds.

25. A method, comprising:
inserting a treatment structure into a cavity corresponding to a tumor bed; and
injecting a radioactive material into the treatment structure whereby the treatment structure takes a natural shape of the cavity, thus substantially mimicking what was removed so that tissue may be built back up to a normal configuration.

26. The method according to claim 25, wherein the radioactive material functions as a radiation carrier, a support for the tumor bed and as a replacement for tissue that is removed to form the tumor bed.

27. The method according to claim 25, wherein the balloon is one of bio-absorbable and non-bio-absorbable, and wherein the radioactive material is one of bio-absorbable and non-bio-absorbable.

28. The method according to claim 25, wherein the radioactive material has at least first and second isotopes, and wherein the method further comprises distributing the first isotope in at least one first area of the tissue surrounding the cavity, and distributing the second isotope in at least one second area of the tissue surrounding the cavity.

29. A method, comprising:
inserting a treatment structure into a cavity corresponding to a tumor bed, the treatment structure taking a natural shape of the cavity, thus substantially mimicking what was removed so that tissue may be built back up to a normal configuration, wherein the treatment structure has a plurality of passages that extend from a common port to a respective plurality of ports at an outer surface of the treatment structure; and
injecting radioactive material into tissue that surrounds the cavity, the radioactive material being introduced to the tissue via the plurality of passages and respective ports.

30. The apparatus according to claim 29, wherein the radioactive material has at least first and second isotopes, and wherein the method further comprises distributing the first isotope in at least one first area of the tissue surrounding the cavity, and distributing the second isotope in at least one second area of the tissue surrounding the cavity.
